(19) United States
(12) Patent Application Publication
(10) Pub. No.: US 2006/0111763 A1
(43) Pub. Date: May 25, 2006

(54) Heat generating article for hyperthermia and method for preparation thereof

(76) Inventors: Tadashi Kokubo, Nagaokakyo-shi (JP); Masahiro Hiraoka, Kyoto-shi (JP); Masakazu Kawashita, Nagaokakyo-shi (JP); Koji Fujita, Chiba-shi (JP); Yasuhiro Saito, Takatsuki-shi (JP); Shinjiro Domi, Abiko-shi (JP)

Correspondence Address: FULBRIGHT & JAWORSKI, LLP, 666 FIFTH AVE, NEW YORK, NY, 10103-3198, US

(21) Appl. No.: 10/525,856

(22) PCT Filed: Aug. 27, 2003
(86) PCT No.: PCT/JP03/10882

§ 371 Date: Oct. 7, 2005

(30) Foreign Application Priority Data
Aug. 29, 2002 (JP) 2002-251764

(51) Int. Cl. A61F 7/00; A61F 7/12

(52) U.S. Cl. 607/96

(57) ABSTRACT

Exothermic elements (30) for hyperthermic treatment having, as a main component thereof, ferromagnetic layers (20) coated outside nucleus particles (10). The ferromagnetic layers (20) comprise an oxide having a magnetic domain structure mainly formed of at least one of a single domain and a pseudo-single domain.

These exothermic elements for hyperthermic treatment may be manufactured by heating after depositing iron hydroxide on the nucleus particles (10) by a liquid phase process.

### TECHNICAL FIELD

The present invention relates to exothermic elements for hyperthermic treatment having ferromagnetic elements as a main component thereof, and a method of manufacturing the same. These exothermic elements for hyperthermic treatment may be used in hyperthermic treatment of cancer.

### BACKGROUND ART

Hyperthermic treatment (hyperthenmia) of cancer has been attracting attention in recent years, which locally warms a tumor portion by using a biological characteristic that cancer tissue is more vulnerable to damage by heat than normal tissue.

In locally warming a tumor portion, attempts have been made to warm the tumor portion from outside the body with warm water, infrared radiation, ultrasonic wave, microwave and so on. With these methods, however, the vicinity of a body surface may be warmed effectively, but it is difficult to warm effectively a deep location in the body without damage to normal tissue.

Lines of magnetic force can be made to reach a deep location in the body without damage to the cells. Taking note of this fact, a proposal has been made to introduce ferromagnetic microspheres into the body with a catheter or the like, place the portion having received the ferromagnetic microspheres in an ac magnetic field, and warm a tumor portion locally by using a generation of heat by hysteresis loss of ferromagnetic microspheres.

A magnetic composition characterized by containing an amorphous alloy and a hydrophilic polymer has been proposed as a magnetic composition suitable for hyperthermia. Cited as examples of this amorphous alloy are one or more of transition metals such as Fe, Ni and Co, one or more of semimetals such as P, C, Si and B, and what contains Cr and/or Mo (see, for example, Patent Publication “Kokai” No. 6-245993; hereinafter referred to as document 1).

A magnetism-sensitive exothermic element having ferric oxide particles of 100-2000 relative permeability as a principal component has also been proposed (see, for example, Patent Publication “Kokai” No. 11-57031; hereinafter referred to as document 2).

As a manufacturing method thereof, a method has been proposed in which magnetite microspheres are prepared from a solution by crystal deposition process (liquid phase process).

Specifically, in the method proposed, silica glass microspheres are immersed as nucleus particles in a hydrofluoric acid solution containing Fe₃O₄ in saturated concentration, to deposit film containing iron, which is heat-treated in a reducing atmosphere gas to obtain microspheres with a diameter of about 25 μm. It has also been reported that the microspheres produced by this method consist of Fe₃O₄ microcrystals with a diameter of about 50 nm, and numerous cracks occur on their surfaces (see, for example, “Chemical Industry Vol. 52, No. 5, (2001) p 38-43”, hereinafter referred to as document 3).

However, the magnetic composition disclosed in the above Document 1 contains an amorphous alloy. Since amorphous generally shows soft magnetism and its hysteresis loss is small, it is unknown whether a sufficient heat generation is secured as exothermic elements for hyperthermic treatment.

Regarding the magnetism-sensitive exothermic elements disclosed in the above document 2, iron oxides such as Fe₃O₄ and γ-Fe₃O₄, spinel type ferric multiple oxides are cited as ferric oxide particles acting as the principal component. However, nothing is disclosed concerning a specific manufacturing method.

Regarding the manufacturing method disclosed in the above document 3, it has been found, as a result of follow-up experiment conducted by Inventors herein based on the description, the deposition reaction of iron hydroxide is unstable, and in some cases, layers of iron hydroxide cannot deposit effectively.

The present invention has been made having regard to the state of the art noted above, and its object is to provide exothermic elements for hyperthermic treatment having high exothermic efficiency, giving sufficient calorific power, and which are magnetically and chemically stable, and a manufacturing method thereof.

### DISCLOSURE OF THE INVENTION

A first characteristic feature of the present invention lies in exothermic elements for hyperthermic treatment having, as a main component thereof, ferromagnetic layers coated outside nucleus particles, wherein said ferromagnetic layers comprise an oxide having a magnetic domain structure mainly formed of at least one of a single domain and a pseudo-single domain.

That is, where the ferromagnetic layers forming the exothermic elements have a magnetic domain structure mainly formed of at least one of a single domain and a pseudo-single domain, the mechanism of flux reversal, when a magnetic field is applied, is not influenced by magnetic walls, e.g. not based on movement of the magnetic walls. Therefore, the energy of the magnetic field is not spent on movement of the magnetic walls, but spent on a generation of heat by magnetic hysteresis loss, thereby increasing the heating value. For example, a sufficient heating value is obtained for treating cancer in the living body. In addition, the ferromagnetic layers consisting of an oxide provides for chemical stability.

A second characteristic feature of the present invention lies in that said ferromagnetic layers substantially consist only of ferromagnetic crystal grains chemically bonded to one another.

That is, since the ferromagnetic layers substantially consist only of ferromagnetic crystal grains chemically bonded to one another, ferromagnetic elements occupy a large proportion, thereby further increasing the heating value. In the absence of a binder component which is an organic substance, chemical stability is secured in the living body.

A third characteristic feature of the present invention lies in that said crystal grains have shape anisotropy.

That is, where the crystal grains have shape anisotropy, their shape effect provides excellent magnetic stability compared with spherical grains. In this specification, the shape having shape anisotropy means shapes other than spherical shape, e.g. approximately rod-like shapes such as cocoon shape, turning ellipsoid and needlelike shapes.

A fourth characteristic feature of the present invention lies in that said ferromagnetic layers have, as a main component thereof, one of gamma hematite, magnetite, strontium ferrite and zinc ferrite.

Where the ferromagnetic layers have, as a main component thereof, one of gamma hematite, magnetite, strontium ferrite and zinc ferrite, it is possible to manufactured easily exothermic elements for hyperthermic treatment from a sufficient heating value can be expected. Since the ferromagnetic layers comprise an inorganic oxide, no harm is done when embedded in the living body, which is desirable.

A fifth characteristic feature of the present invention lies in that said exothermic elements are spherical or approximately spherical, and 10 to 200 μm in diameter.

That is, the exothermic elements which are spherical or approximately spherical, and 10 to 200 μm in diameter, are convenient to embed and fix in a blood vessel of the living body, for example.

Particularly, the exothermic elements 10 to 40 μm in diameter may be embedded and fixed in a peripheral blood vessel of the living body, for example. This is advantageous in providing a hyperthermic effect and an embolus effect for a tumor.

A sixth characteristic feature of the present invention lies in that said ferromagnetic layers have a volume ratio at least 3.5 times said nucleus particles.

Where the ferromagnetic layers have a volume ratio at least 3.5 times the nucleus particles, the ferromagnetic layers have an increased proportion, and a sufficient heating value may be expected.

A seventh characteristic feature of the present invention lies in that said ferromagnetic layers have cracks formed therein, said cracks having a maximum width corresponding to at most 3% of diameters of said exothermic elements.

Where the cracks formed in the ferromagnetic layers have a maximum width corresponding to at most 3% of diameters of the exothermic elements, even if cracks are formed in the ferromagnetic layers, the cracks are small relative to the exothermic elements, whereby fragmentation of part of the ferromagnetic layers is restrained. It is therefore desirable that fragmented parts of the ferromagnetic layers have a reduced chance of moving away from a location under treatment.

An eighth characteristic feature of the present invention lies in that said nucleus particles have a mean diameter of 0.1 to 10 μm, a coefficient of variation in particle size thereof being at most 15%.

That is, by using nucleus particles of such mean particle diameter, spherical or approximately spherical exothermic elements 20 to 40 μm in diameter may be obtained easily. Further, where nucleus particles having a coefficient of variation in particle size being at most 15% are used, exothermic elements of small dispersion in particle size may be obtained easily.

A ninth characteristic feature of the present invention lies in that said nucleus particles are formed of silicon oxide.

Where the nucleus particles are formed of silicon oxide, the ferromagnetic layers may be formed on the nucleus particles with sufficient adhesion thereto by liquid phase process. The ferromagnetic layers are prevented from peeling and forming large cracks. Chemical stability also is secured advantageously.

A tenth characteristic feature of the present invention lies in that metal oxide thin film is coated on surfaces of the exothermic elements.

Where the surfaces of the ferromagnetic layers are covered with metal oxide thin film, the metal oxide thin film acts as a protective film. Even when cracks occur in the ferromagnetic layers, fragmentation of the ferromagnetic layers is prevented. It is therefore advantageous in that, when the exothermic elements are embedded in the living body to be used for hyperthermic treatment, the ferromagnetic layers have a reduced chance of fragmenting and moving away from a location under treatment. It is also advantageous that this thin film is a metal oxide, which is chemically stable.

An eleventh characteristic feature of the present invention lies in that said metal oxide thin film is formed of one of silicon oxide, titanium oxide, gamma hematite, magnetite and iron hydroxide.

It is advantageous in that the metal oxide thin film formed of one of silicon oxide, titanium oxide, gamma hematite, magnetite and iron hydroxide is not rejected by the living body since these substances have affinity with the living body. Further, the metal oxide thin film, when formed of gamma hematite or magnetite, also contributes to heating, which is advantageous.

A twelfth characteristic feature of the present invention lies in that said metal oxide thin film is porous.

Where the metal oxide thin film is porous, the metal oxide thin film may be medicated. This is advantageous in that the hyperthermic treatment may be carried out in parallel with treatment with a medicinal substance.

A thirteenth characteristic feature of the present invention lies in that the exothermic elements for hyperthermic treatment comprise only an inorganic material.

The exothermic elements comprising only an inorganic material has an advantage of being chemically stable.

A fourteenth characteristic feature of the present invention lies in that said exothermic elements have a heating value of 5 to 30 [W/g] when placed in an ac magnetic field with a frequency of 100 kHz and at 15.92 to 29.45 [kA/m].

The heating value of 5 to 30 [W/g] has an advantage of being sufficient for hyperthermic treatment, and capable of effective hyperthermic treatment.

A fifteenth characteristic feature of the present invention lies in a method of manufacturing exothermic elements for hyperthermic treatment having, as a main component thereof, ferromagnetic layers coated outside nucleus particles, wherein, after performing a deposition treatment for depositing and forming layers of iron hydroxide around said nucleus particles by a liquid phase process, a heating treatment is performed in a reducing atmosphere to change the iron hydroxide layers formed around said nucleus particles to a ferromagnetic material comprising gamma hematite, to form said ferromagnetic layers.

In such a method of manufacturing exothermic elements for hyperthermic treatment, a deposition treatment is first performed for depositing and forming layers of iron hydroxide around the nucleus particles by a liquid phase process, whereby iron hydroxide is deposited uniformly around the nucleus particles. Then, a heating treatment is performed in a reducing atmosphere to form ferromagnetic layers comprising gamma hematite.

According to this manufacturing method, exothermic elements for hyperthermic treatment comprise ferromagnetic layers having a magnetic domain structure mainly formed of at least one of a single domain and a pseudo-single domain. This is a method capable of forming homogeneous ferromagnetic layers on the nucleus particles, and manufacturing exothermic elements for hyperthermic treatment in large quantities and economically.

A sixteenth characteristic feature of the present invention lies in that, in the above manufacturing method, said nucleus particles have a mean diameter of 0.1 to 10 μm, a coefficient of variation in particle size thereof being at most 15%.

Where such nucleus particles are used, since their dispersion in particle size is small, exothermic elements for hyperthermic treatment with a uniform particle size may be manufactured reliably.

A seventeenth characteristic feature of the present invention lies in that, in the above manufacturing method, said heating treatment is carried out at a heating rate of at most 5° C./min. within a range of 100 to 500° C.

Such a heating treatment can restrain a concentration on a certain particular part of a stress due to a volume shrinkage caused by a dehydration condensation reaction of the ferromagnetic layers. This provides an advantage of reducing the widths of cracks formed.

An eighteenth characteristic feature of the present invention lies in that, in the above manufacturing method, said heating rate is at most 1° C./min.

Such a heating treatment has an advantage of further reducing the widths of cracks formed.

A nineteenth characteristic feature of the present invention lies in that, in the above manufacturing method, said heating treatment is carried out to reduce said iron hydroxide layers by placing and rotating, in a cylindrical drum, said nucleus particles with said iron hydroxide layers formed thereon.

Such a heating treatment has an advantage of uniformly reducing the nucleus particles with the iron hydroxide layers thereon.

Generally, a minimum magnet unit inside a ferromagnetic element is called a magnetic domain. A structure having only one magnetic domain in one crystal grain is called a single domain structure. A structure having a plurality of magnetic domains is called a multi-magnetic domain structure. In this specification, a pseudo-single domain is defined as having several (2 to 5) magnetic domains in one crystal grain.

The coefficient of variation means a ratio between population standard deviation σ and population mean μ of a population, which is expressed by the following equation:
Coefficient of variation CV=σ/μ×100 [%]

### BRIEF DESCRIPTION OF THE DRAWINGS

FIG. 1 is a schematic view of a film depositing apparatus for forming iron hydroxide layers;

FIG. 2 is a schematic view of a reducing furnace;

FIG. 3 is a view tracing outlines of crystal grains constituting ferromagnetic layers; and

FIG. 4 is a schematic sectional view of an exothermic element for hyperthermic treatment.

### BEST MODE FOR CARRYING OUT THE INVENTION

Embodiments of exothermic elements for hyperthermic treatment and manufacturing methods thereof according to the present invention will be described hereinafter.

### Embodiment 1

A deposition treatment was carried out as follows to deposit iron hydroxide, by liquid phase process, to form iron hydroxide layers around spherical silica particles which were an example of nucleus particles. Further, by heating these, the iron hydroxide layers were changed to gamma hematite to form the ferromagnetic layers, thereby preparing exothermic elements for hyperthermic treatment according to the present invention.

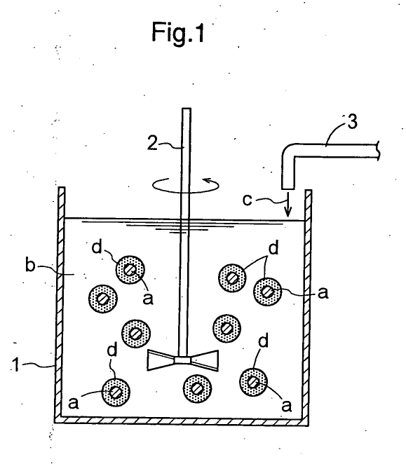

First, 50 mL of hydrofluoric acid of 0.1 mol/L was added to 1 L of an aqueous solution of ferric fluoride (FeF₃) of 0.5 mol/L to prepare a treating solution for the deposition treatment. This treating solution, 1 L, was put into a water bath, at 35° C., and 0.6 g of spherical silica particles with a particle size of about 9 μm (manufactured by Admatechs Co., Ltd.) and fully dried beforehand were immersed as the nucleus particle in the treating solution. Then, an aqueous solution of boric acid (H₃BO₃) of 0.5 mol/L was dripped as a reaction initiator to this treating solution. The solution was allowed to react under an agitated condition for 30 days, to deposit β-FeOOH which was an example of iron hydroxide around the spherical silica particles (see FIG. 1).

FIG. 1 is a schematic view of an apparatus for depositing iron hydroxide on the nucleus particles by liquid phase process to form iron hydroxide layers thereon. In the figure, ‘a’ denotes the spherical silica particles which are an example of nucleus particles, ‘b’ denotes the treating solution, ‘c’ denotes the aqueous solution of boric acid which is an example of reaction initiators, ‘d’ denotes β-FeOOH which is an example of iron hydroxide, 1 denotes a container, 2 a stirrer, and 3 a pipe for supplying the reaction initiator.

FIG. 1 shows a state in which the spherical silica particles ‘a’ are put into the treating solution ‘b’, and while dripping the aqueous solution of boric acid ‘c’, the treating solution ‘b’ is agitated by the stirrer 2.

The entirety of the treating solution was changed to a new one every 10 days. And after 30 days microspheres of β-FeOOH deposited around the spherical silica particles were taken out of the treating solution, fully cleaned and then dried at 100° C.

These microspheres were heated at a rate of 5° C./min. from room temperature under a reducing atmosphere of a mixed gas (at a total flow rate of 100 mL/min.) with a volume ratio of CO₂ to H₂ (CO₂:H₂) at 70:30, heated at 600° C. for one hour, and then allowed to cool.

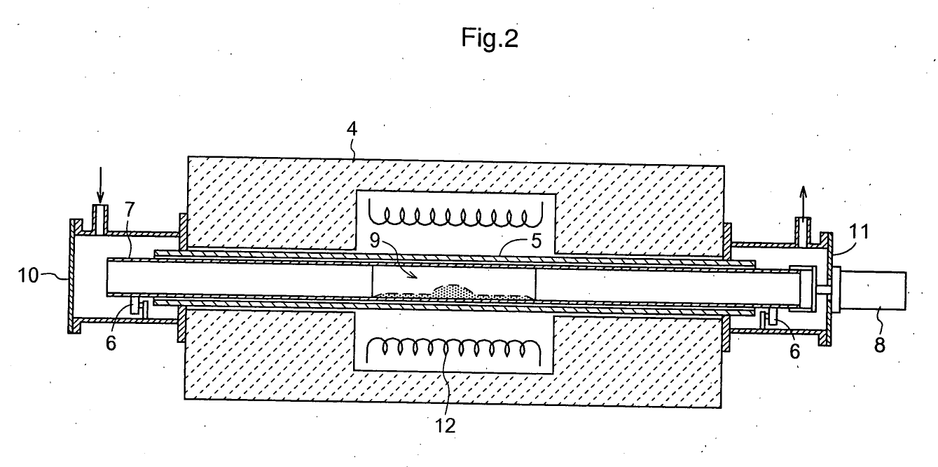

FIG. 2 shows a reducing furnace used in this heating treatment, which includes a heating furnace 4, a furnace tube 5 formed of quartz and disposed centrally of the heating furnace 4, a rotating pipe 7 formed of quartz and rotatable inside the furnace tube 5 as a tubular drum, and a motor 8 for driving and rotating the rotating pipe 7. The rotating pipe 7 is rotatably supported at opposite ends thereof by a plurality of rollers 6. A sample chamber 9 is formed inside the rotating pipe 7, and a heater 12 is disposed to surround the sample chamber 9.

Flanges 10 and 11 are arranged at opposite ends of the furnace tube 5 for maintaining a reducing atmosphere therein. While introducing a reducing gas from one flange 10 and discharging it from the other flange 11, heating treatment is carried out in the reducing atmosphere.

Such a reducing furnace is used, and the microspheres covered with β-FeOOH are put into the sample chamber 9 of the rotating pipe 7. The flanges 10 and 11 are closed tight, a reducing gas is introduced to produce a reducing atmosphere, and heating treatment is performed while rotating the rotating pipe 7. Then, a uniform reducing effect is advantageously obtained.

In this way, exothermic elements for hyperthermic treatment were obtained, which had ferromagnetic layers of gamma hematite changed from β-FeOOH. That is, the exothermic elements for hyperthermic treatment have, as a main component thereof, gamma hematite layers covering the outside of the spherical silica particles which are nucleus particles. The exothermic elements for hyperthermic treatment according to the present invention have the ferromagnetic layers as a main component thereof which constitutes at least more than half of the exothermic elements in volume. It is preferred that the ferromagnetic layers form 80% or more of the exothermic elements.

It has been confirmed by X-ray diffraction method that the ferromagnetic layers of the exothermic elements has gamma hematite as a principal component. An observation with a scanning electron microscope (SEM) shows that the exothermic elements are about 25 μm in diameter, and that the gamma hematite layers are 8 μm thick. Further, it has been observed that the ferromagnetic layers of the exothermic elements are porous.

And the magnetic domain structure of the gamma hematite layers of these exothermic elements was checked. The magnetic domain structure was checked by using a scanning probe microscope (SPM; SPI3700 manufactured by Seiko Instruments Inc.(Sll)) as a magnetic force microscope (MFM). It has been found as a result that magnetic domain size is about 40 nm, and it is certainly a single magnetic domain structure.

The magnetic domain structure of the ferromagnetic layers having a single magnetic domain or pseudo-single magnetic domain, as noted above, provides a large hysteresis loss, which is desirable as the exothermic elements for hyperthermic treatment.

A magnetic domain observation may be carried out with a spectral domain viewing device, domain scope, high magnetic field microcar effect measuring device as well.

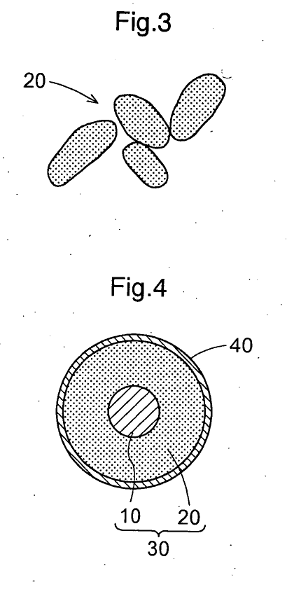

Further, the ferromagnetic layers of the exothermic elements were observed with a transmission electron microscope (TEM). FIG. 3 shows outlines of crystal grains constituting the ferromagnetic layers, which are schematically traced from the results. It has been found therefrom that the crystal grains are cocoon-shaped and has shape anisotropy.

Thus, the ferromagnetic layers which are aggregates of crystal grains having shape anisotropy are magnetically stable and desirable.

The ferromagnetic layers consist only of crystal grains of ferromagnetic elements substantially chemically bonded to one another, without binder component. Such ferromagnetic layers are desirable in having an increased ratio in the exothermic elements for hyperthermic treatment. In addition, the exothermic elements for hyperthermic treatment may be formed only of an inorganic material, and thus have excellent chemical stability also.

With the embodiments shown hereinafter included, uniform exothermic elements with a reduced dispersion in particle size have been obtained by using nucleus particles having a reduced dispersion in particle size, and forming ferromagnetic layers by liquid phase process.

### Embodiment 2

Exothermic elements for hyperthermic treatment with the surfaces of exothermic elements covered by silicon dioxide thin film which is an example of metal oxide thin film were prepared by the following method.

A SiO₂ saturated aqueous solution having hydrofluoric acid and silica gel in the state of equilibrium was prepared by dissolving silica gel in silicofluoric acid of 2.5 mol/L. Further, four 50×50×3mm aluminum plates were immersed in 1 L of this SiO₂ saturated aqueous solution for one hour to produce a SiO₂ supersaturation aqueous solution. The quantity of aluminum dissolved at this time was 2.6 g/L. This 1 L of SiO₂ supersaturation aqueous solution was put into a water bath at 35° C., and 1.5 g of microspheres covered with gamma hematite layers fully cleaned and dried beforehand were immersed as exothermic elements in the aqueous solution. The solution was allowed to react under an agitated condition for five hours, to deposit silicon dioxide thin film on the surfaces of the gamma hematite layers.

Thereafter, these microspheres were taken out of the supersaturated aqueous solution, fully cleaned and then dried at 100° C.

By repeating the above operation four times, exothermic elements for hyperthermic treatment having, as a main component, the gamma hematite layers with silicon dioxide thin film coated on the surfaces thereof were obtained.

These exothermic elements were subjected to a composition analysis by fluorescent X-ray method. It has been found as a result that the silicon dioxide thin film is coated on the surfaces of the gamma hematite layers. Further, when sections and surfaces of the exothermic elements were observed with a scanning electron microscope, it was found that the thickness of the silicon dioxide thin film was about 100 nm, and that the surfaces were porous.

FIG. 4 shows a sectional structure of an exothermic element 30 for hyperthermic treatment, showing a state that the exothermic element 30 includes a nucleus particle 10, and a ferromagnetic layer 20 coated therearound, with the surface covered by a thin film 40.

### Embodiment 3

Exothermic elements for hyperthermic treatment with the surfaces of exothermic elements covered by iron hydroxide thin film which is an example of metal oxide thin film were prepared by the following method.

First, a treating solution for deposition treatment was prepared by adding 50 mL of hydrofluorc acid of 0.1 mol/L to 1 L of an aqueous solution of ferric fluoride of 0.5 mol/L. This 1 L of treating solution is put into a water bath at 35° C., and 0.6 g of the exothermic elements with a particle diameter of about 25 μm prepared in Embodiment 1 were immersed in the treating solution. Subsequently, an aqueous solution of boric acid of 0.5 mol/L was dripped to this treating solution. The solution was allowed to react under an agitated condition for 30 days, to deposit β-FeOOH which was an example of iron hydroxide around the exothermic elements

These exothermic elements were subjected to a composition analysis by fluorescent X-ray method. It has been found as a result that the iron hydroxide thin film is coated on the surfaces of the ferromagnetic layers. Further, when sections and surfaces of the particles were observed with a scanning electron microscope, it was found that the thickness of the iron hydroxide thin film was about 500 nm.

Where the surfaces of the ferromagnetic layers of the exothermic elements are covered with silicon dioxide thin film or iron hydroxide thin film as described above, the ferromagnetic layers are prevented from becoming fragmented. Thus, the exothermic elements for hyperthermic treatment may be maintained in an intended location in the living body. Further, these exothermic elements for hyperthermic treatment have biocompatibility, which is desirable.

Various experiments and observations have been conducted to check the effects and the like of the present invention, and their results will be discussed next.

(Experiment 1)

Since measurement of heating values of the exothermic elements in the living body involves difficulties, the heating values of the exothermic elements were measured by the methods shown below.

A sample for thermometry was prepared by adding 0.1 g of the exothermic elements obtained in Embodiment 1 to an aqueous solution of agar with 0.2 g of agar dissolved in 20 ml of hot water at 100° C., dispersing them uniformly by using an ultrasonic wave, and then cooling and solidifying this aqueous solution.

This sample was fully insulated with styrene foam, and placed in an AC magnetic field with a frequency of 100 kHz and at 23.88 kA/m (300 [Oe]). The state of the magnetic field was measured with a gauss meter (HGM-7500 S-type) manufactured by ADS Co., Ltd. The temperature rise of the sample after applying the ac magnetic field for 10 minutes was about 13° C. Therefore, the heating value of the exothermic elements is presumed to be 18 W/g.

Similar experiment was conducted by varying the magnetic field, and the results are shown in Table 1 below.

TABLE 1

| | field applied | temperature rise | Heating value |
|---|---|---|---|
| frequency (kHz) | (kA/m) (Oe) | (° C.) | (W/g) |
| 100 | 15.92 (200) | 4 | 6 |
| 100 | 19.90 (250) | 7 | 10 |
| 100 | 23.88 (300) | 13 | 18 |
| 100 | 25.45 (370) | 19 | 26 |

(Experiment 2)

The surface area of the sample prepared in Embodiment 1 (hereinafter Sample 1) measured by BET method was about 5 m²/g. The surface area of the silica particles of the same particle size was about 0.2 m²/g. Compared with these, sample 1 had a surface area 10 times or more.

When cracks in particle surfaces of Sample 1 were observed with a scanning electron microscope, a maximum width of the cracks was 0.5 μm.

(Experiment 3)

Microspheres with β-FeOOH deposited therearound were obtained as in Embodiment 1. These were heated at 600° C. for one hour at a rate of 1° C. /min. from room temperature and were allowed to cool under a reducing atmosphere of a mixed gas of CO₂ and H₂, to change β-FeOOH to gamma hematite layers. As a result, Sample 2 was obtained, which comprised exothermic elements with spherical silica particles covered by the gamma hematite layers (ferromagnetic layers).

When cracks in the surfaces of the particles of Sample 2 were observed with a scanning electron microscope, a maximum crack width was 0.2 μm.

The following comparative experiment was conducted in order to observe differences in crack width due to heating rates.

(Comparative Experiment)

Microspheres with β-FeOOH deposited therearound were obtained as in Embodiment 1 and Experiment 3. These were heated at 600° C. for one hour at a rate of 10° C./min. from room temperature and were allowed to cool under a reducing atmosphere of a mixed gas of CO₂ and H₂, to change β-FeOOH to gamma hematite layers. As a result, Sample 3 was obtained, which comprised exothermic elements with spherical silica particles covered by the gamma hematite layers (ferromagnetic layers).

When cracks in the surfaces of the particles of Sample 3 were observed with a scanning electron microscope, a maximum crack width was 1.5 μm.

Table 2 shows these results in summary.

TABLE 2

| | heating rate | grain size | max. crack | crack/grain size |
|---|---|---|---|---|
| | (° C./min) | (μm) | width (μm) | (%) |
| Embodiment 1 | 5 | 2.5 | 0.5 | 2 |
| Experiment 3 | 1 | 2.5 | 0.2 | 0.8 |
| Comp. | 10 | 2.5 | 1.5 | 6 |
| experiment | | | | |

Each of Samples 1, 2 and 3 was placed in about 0.1 g of physiological saline, which was agitated intensely for one minute with a homogenizer. When the particles were collected and observed with an optical microscope, Samples 1 and 2 showed no peeling of the ferromagnetic layers, but Sample 3 clearly showed peeling.

Embodiment 1 to Embodiment 3 and Experiment 3 described above are examples of the present invention, and the present invention is not limited to these embodiments. Other embodiments will be described hereinafter.

### Other Embodiments

<1> The exothermic elements for hyperthermic treatment according to the present invention may only have ferromagnetic elements as the main component, and are not limited to the exothermic elements illustrated hereinbefore. Further, the exothermic elements may have a magnetic domain structure mainly comprising at least one of the single domain and pseudo-single domain.

The ferromagnetic layers may only be formed of aggregates of crystal grains having oxide shape anisotropy. That is, the crystal grains, instead of being spherical, may have shape anisotropy such as cocoon-shaped, turning ellipsoid or needlelike.

Such exothermic elements, when manufactured by the method of using a balanced reaction of ferric fluoride ions as illustrated in foregoing Embodiment 1, may be deposited stably, have excellent controllability of the reaction, and have the nucleus particles uniformly covered with iron hydroxide layers. These are desirable since the diameter of crystal deposits may be controlled with ease.

The exothermic elements may be manufactured by other liquid phase processes such as a method of neutralizing aqueous acids containing iron ions. The exothermic elements are not limited to the methods described above. For example, by a nonelectrolytic plating method using a solution containing at least iron ions and a reducing agent, layers of various ferrites further to gamma hematite and magnetite may be formed directly.

<2> The exothermic elements are not limited to what comprises gamma hematite illustrated hereinbefore, but may be formed of various ferromagnetic materials. For example, they may be formed of one or more ferromagnetic materials selected from magnetite, strontium ferrite and zinc ferrite.

<3> As the metal oxide thin film, the example in Embodiment 2 has been described as comprising silicon dioxide, and the example in Embodiment 3 as comprising iron hydroxide. It may comprise titanium oxide, magnetite or the like. Where the metal oxide thin film comprises these materials, as in the case of silicon dioxide or iron hydroxide, fragmentation of gamma hematite particles which are the exothermic elements can be prevented. Thus, the exothermic elements for hyperthermic treatment may be maintained in an intended location in the living body. Further, these exothermic elements for hyperthermic treatment have biocompatibility, which is desirable.

Oxides of various metals other than these, as long as they are materials having chemical stability, may be used as materials for the metal oxide thin film. As the method of forming the metal oxide thin film, as illustrated in the above embodiments, the liquid phase process is preferred.

<4> While the exothermic elements are not be limited to a particular form as long as they are the form of particles showing ferromagnetism, spherical or approximately spherical form is preferred since the exothermic elements for hyperthermic treatment may then be brought to an intended location in the living body with ease. The exothermic elements, most desirably, are 10 to 40 μm in diameter in order to stop in a peripheral part of an artery feeding a tumor, without passing through capillary vessels, to demonstrate an embolus effect.

In order to obtain spherical or approximately spherical exothermic elements, the liquid phase process may be applied to spherical or approximately spherical nucleus particles. Then, exothermic elements of uniform particle size may be obtained easily.

The shape of nucleus particles is not limited to spherical, but those of any available shape may be used. However, the shape of the particles with deposits thereon of the iron hydroxide layers obtained by the method shown in above Embodiment 1 well reflects the shape of the nucleus particles. Further, the shape of the exothermic elements after the heating treatment also well reflects the shape of the nucleus particles. Therefore, the shape of nucleus particles may be spherical, by taking the embolus effect into account, when spherical exothermic elements are to be obtained.

Nucleus particles, spherical, having a diameter of 0.1 to 10 μm, and with a coefficient of variation in particle size being 15% or less, are particularly desirable in that exothermic elements of uniform particle size may be obtained.

The above embodiments have shown an example using spherical silica particles as nucleus particles. The material for nucleus particles is not limited to this, as long as the particles have excellent dispersibility or chemical stability in the treating solution for depositing iron hydroxide. It is advantageous to use a material showing ferromagnetism as nucleus particles, since the exothermic elements for hyperthermic treatment are then entirely formed of ferromagnetic materials, and increased heating values are expected therefrom.

As described above, desirable nucleus particles in the present invention are spherical particles Such as of silicon dioxide or titanium dioxide. Of these, since silicon dioxide particles are preferred since a uniform particle size is obtained easily by methods Such as liquid phase deposition which counteracts an aqueous solution of soda silicate, and a sol gel process which uses tetra-ethoxy silane as the starting material.

<5> The surfaces of the exothermic elements are not limited to the above-noted metal oxide thin film, but may be an active inorganic material having affinity with bones and body tissues, such as hydroxylapatite.

<6> The exothermic elements for hyperthermic treatment according to the present invention may also be guided to a desired location in the body to be treated, by moving magnetic fields from outside.

### INDUSTRIAL UTILITY

The exothermic elements for hyperthermic treatment according to the present invention may be used for hyperthermic treatment of cancer. Instead of being limited to this, they may be used in various other applications, for locally warming affected parts.

1. Exothermic elements for hyperthermic treatment comprising ferromagnetic layers coated outside nucleus particles,
wherein said ferromagnetic layers comprise an oxide having a magnetic domain structure mainly formed of at least one of a single domain and a pseudo-single domain.

2. Exothermic elements for hyperthermic treatment as defined in claim 1, wherein said ferromagnetic layers consist essentially of ferromagnetic crystal grains chemically bonded to one another.

3. Exothermic elements for hyperthermic treatment as defined in claim 2, wherein said crystal grains have shape anisotropy.

4. Exothermic elements for hyperthermic treatment as defined in claim 1, wherein said ferromagnetic layers comprise gamma hematite, magnetite, strontium ferrite or zinc ferrite.

5. Exothermic elements for hyperthermic treatment as defined in claim 1, wherein said exothermic elements are spherical or approximately spherical, and 10 to 200 μm in diameter.

6. Exothermic elements for hyperthermic treatment as defined in claim 1, wherein said ferromagnetic layers have a volume ratio at least 3.5 times said nucleus particles.

7. Exothermic elements for hyperthermic treatment as defined in claim 5, wherein said ferromagnetic layers have cracks formed therein, said cracks having a maximum width corresponding to at most 3% of diameters of said exothermic elements.

8. Exothermic elements for hyperthermic treatment as defined in claim 5, wherein said nucleus particles have a mean diameter of 0.1 to 10 μm, and have a coefficient of variation in particle size thereof being at most 15%.

9. Exothermic elements for hyperthermic treatment as defined in claim 1, wherein said nucleus particles are formed of silicon oxide.

10. Exothermic elements for hyperthermic treatment as defined in claim 1, comprising metal oxide thin film coated on surfaces of said exothermic elements.

11. Exothermic elements for hyperthermic treatment as defined in claim 10, wherein said metal oxide thin film comprises silicon oxide, titanium oxide, gamma hematite, magnetite or iron hydroxide.

12. Exothermic elements for hyperthermic treatment as defined in claim 10, wherein said metal oxide thin film is porous.

13. Exothermic elements for hyperthermic treatment as defined in claim 1, comprising only an inorganic material.

14. Exothermic elements for hyperthermic treatment as defined in claim 1, wherein said exothermic elements have a heating value of 5 to 30 [W/g] when placed in an AC magnetic field with a frequency of 100 kHz and at 15.92 to 29.45 [kA/m].

15. A method of manufacturing exothermic elements for hyperthermic treatment wherein said exothermic elements comprise ferromagnetic layers coated outside nucleus particles, comprising:
performing a deposition treatment for depositing and forming layers of iron hydroxide around said nucleus particles by a liquid phase process,
performing a heating treatment in a reducing atmosphere to change the iron hydroxide layers formed around said nucleus particles to a ferromagnetic material comprising gamma hematite, to form said ferromagnetic layers,
whereby said ferromagnetic layers comprise an oxide having a magnetic domain structure mainly formed of at least one of a single domain and a pseudo-single domain.

16. A method of manufacturing exothermic elements for hyperthermic treatment as defined in claim 15, wherein said nucleus particles have a mean diameter of 0.1 to 10 μm, and have a coefficient of variation in particle size thereof being at most 15%.

17. A method of manufacturing exothermic elements for hyperthermic treatment as defined in claim 15, wherein said heating treatment is carried out at a heating rate of at most 5° C./min. within a range of 100 to 500° C.

18. A method of manufacturing exothermic elements for hyperthermic treatment as defined in claim 17, wherein said heating rate is at most 1° C./min.

19. A method of manufacturing exothermic elements for hyperthermic treatment as defined in claim 15, wherein said heating treatment is carried out to reduce said iron hydroxide layers by placing and rotating, in a cylindrical drum, said nucleus particles with said iron hydroxide layers formed thereon.
